(19) United States
(12) Patent Application Publication
(10) Pub. No.: US 2018/0263632 A1
(43) Pub. Date: Sep. 20, 2018

(54) DEVICE AND METHOD FOR ENDOVASCULAR TREATMENT OF ANEURYSMS USING EMBOLIC ePTFE

(71) Applicants: Paul Steven SEIFERT, (US); Adam Immanuel SEIFERT, (US); Neuravi Limited, Galway (IE)

(72) Inventors: Paul Steven SEIFERT, Oregon House, CA (US); Adam Immanuel SEIFERT, Oregon House, CA (US); David VALE, County Galway (IE); Michael GILVARRY, County Galway (IE); Brendan CASEY, Galway (IE); Brian FAHY, County Galway (IE); Jacqueline O'GORMAN, County Clare (IE)

(21) Appl. No.: 15/985,499

(22) Filed: May 21, 2018

Related U.S. Application Data
(63) Continuation of application No. 14/690,984, filed on Apr. 20, 2015.
(60) Provisional application No. 61/966,287, filed on Feb. 21, 2014.

(51) Int. Cl. A61B 17/12; A61L 31/10; A61L 31/02; A61L 31/18; A61L 31/14; A61L 31/04; A61B 17/00

(52) U.S. Cl. CPC A61B 17/12113; A61L 31/10; A61B 17/1215; A61L 31/022; A61L 31/18; A61L 2430/36; A61B 17/12145; A61L 31/048; A61B 2017/00831; A61L 2400/16; A61L 31/146

(57) ABSTRACT

An embolic occlusion device for the treatment of aneurysms and arteriovenous malformations comprises expanded polytetrafluoroethylene (ePTFE). The ePTFE permits ingrowth of cells with connective tissue deposition to promote adherence of the aneurysm wall to the embolic device thereby preventing continued growth or re-growth of the aneurysm as well as blocking blood flow into an aneurysm. An occlusion device is also described which comprises an embolic element and a polymeric pre-formed component.

### CROSS REFERENCE TO RELATED APPLICATIONS

This application is a continuation of U.S. application Ser. No. 14/690,984, filed on Apr. 20, 2015, which claims the benefit of U.S. Provisional Application No. 61/966,287, filed Feb. 21, 2014. The entire contents of which are incorporated herein by reference.

### FIELD OF THE DISCLOSURE

This disclosure provides a method and a device for treating aneurysms in the intracranial and extracranial circulation, or aneurysms found in other parts of the body. Treated aneurysms may be ruptured or unruptured. Aneurysm rupture can lead to debilitating or life threatening events and the disclosure described herein represents a treatment modality to inhibit or prevent aneurysm rupture or hemorrhaging from a ruptured aneurysm. It can also be applied to any condition that would benefit from target specific occlusion.

### BACKGROUND

Cerebral aneurysms are the most common cause of subarachnoid hemorrhage (stroke) and there rupture can result in morbidity and mortality. Hemorrhaging within the brain is of considerable concern since it may result in loss of central nervous tissue functioning. Microsurgical clipping of aneurysms is an effective means of treatment; however it is an invasive surgical procedure and therefore exhibits attendant risks and recovery issues. Clipping as a therapeutic approach has now been largely been replaced by less invasive intravascular methods, at least for aneurysms less than 10 millimeters in diameter. Intravascular treatment methods involve catheter based delivery systems for placing embolic coils (e.g. platinum wires) within the aneurysm. The embolic coils fill the aneurysm space thereby inhibiting the inflow of blood from the parent artery. The coil wires typically form a semi-controlled tangled mass of metal, or other material, within the aneurysm sac thereby attenuating blood flow into the aneurysm and providing it with protection from rupture and possibly further growth. Simply put, embolic coils are a form of (semi) occlusive plug that redirects blood flow away from the aneurysm opening thereby either preventing hemorrhage or possibly even sealing off the aneurysm opening.

A variety of coil shapes and configurations have been invented. For example, U.S. Pat. No. 5,624,461 describes a three-dimensional occlusive coil and U.S. Pat. No. 5,639,277 describes embolic coils with twisted helical shapes. Spherical shaped embolic coil devices were described in U.S. Pat. No. 5,645,558 where one or more strands are wound to form a substantially hollow spherical or ovoid shape when deployed. The embolic coils can be detached from their introducer wire using mechanical means (U.S. Pat. No. 5,234,437) or electrolytically (U.S. Pat. No. 5,122,136).

In addition to coil wires, various coating materials have been introduced that are variably intended to either provide superior filling capacity or bioactive properties. Such coatings included hydrogels and bioresorbable polymers like polygylcolic or polylactic acids (or co-polymers thereof). Even cyanoacrylate resins and glues have been assessed for aneurysm closure.

The use of embolic coils for more than a decade has demonstrated their usefulness in treating cerebral aneurysms. Nevertheless they exhibit deficiencies. Prominent among them is compaction of the coils leading to aneurysm regrowth. Failure to adequately impede blow flow into the aneurysm and consequent aneurysm perpetuation places the patient at risk for stroke. Indeed, histological evidence from clinically derived specimens at autopsy demonstrates that the aneurysm sac inadequately heals.

In attempts to improve aneurysm healing, embolic coil wires have been coated with various materials. In one such aspect, platinum wires have been coated with a bioresorbable suture material. However, clinical studies have shown this improvement to be ineffective. Other coatings (e.g. proteins and polyurethanes) have certain disadvantages as well such as reducing the deliverability and overall performance within the microcatheter system or sloughing off of coating materials during transit to the treatment site.

### STATEMENTS OF DISCLOSURE

According to the disclosure there is provided an intravascular embolic device that fills the aneurysmal space thereby blocking blood flow into the aneurysm and resulting over time to tissue in-filling to protect it from regrowth or rupture.

The device may be composed of a core wire, (e.g. platinum, nickel alloy, biodegradable material) to which ePTFE is a fixed to its outer surface. The ePTFE exhibits a pore structure (e.g. 10¬60 um or a fibrillar tangle) that enables cells to migrate into it. The ePTFE may be in the form of a ribbon or tape that is adhered to the core wire using an appropriate adhesive or other fixation process. The surface exposed to blood is therefore ePTFE that is supported by the wire core. This ePTFE coil may be delivered to the aneurysm using a catheter-based system and detachment of the aneurysm occlusion device (i.e. the ePTFE coated coil wire) from the transport catheter may be achieved by mechanical, electrical and/or chemical decoupling.

In one embodiment the coil may be composed of ePTFE polymer with a radio-opaque material disposed thereon.

In one embodiment, the coil core wire is a bioresorbable polymer with radio-opaque material and the resorbable coil is coated with ePTFE polymer.

In one embodiment the interstices of the ePTFE polymer may be loaded with a biologically active agents such, but not limited to, growth factors [e.g. vascular endothelial growth factor (VEGF), a basic fibroblast growth factor (bFGF), platelet derived growth factor (PDGF)]; tissue factor and/or; cytokines.

According to the disclosure there is provided a system for the treatment of aneurysms, the system comprising:—

an aneurysm filling component and a delivery device;
the aneurysm filling component comprising a polymeric component;
the polymeric component having a cohesive energy density of less than 60 cal/cm³ and an internodal distance of less than 200 microns;
the delivery device comprising a catheter;
and the aneurysm filling component configured to be delivered through the catheter to the target aneurysm.

The aneurysm filling component may further comprise a structural component. This structural component may comprise one or more wire components. One or more of these components may be formed into a coil. One or more of these wire components may be formed from Stainless Steel or Nitinol or other metal or a radiopaque metal such as Platinum or Gold or other radiopaque material an alloy of one of these. The coil structure may in turn be formed into a tertiary shape that the aneurysm filling component preferentially adopts when in the freely expanded state. Such a tertiary shape may comprise a three dimensional structure that is somewhat spherical or cylindrical or conical or other such shape that assists the aneurysm filling component in filling the internal space within the aneurysm.

In one embodiment the polymeric component is a coating or layer covering or integrated into the structural component.

In one embodiment the cohesive energy density of the polymeric component is less than 50 cal/cm³ and in a further embodiment the cohesive energy density is less than 40 cal/cm³.

In one embodiment the polymeric component is ePTFE.

The polymeric component may be in the form of a tube, and in one embodiment the tube is further processed to remove discrete sections of material. The polymeric component may alternatively be in the form of a coating or a tape or strands or filaments, and may be wrapped around or through a substrate material such as the structural component.

In one embodiment the polymeric component is a tape that is wrapped around a wire, and the wire is in turn wrapped into a coil structure. The coil structure may in turn be formed into a tertiary shape that the aneurysm filling component preferentially adopts when in the freely expanded state.

In one embodiment the polymeric component is a tape that is wrapped around a coil structure such that the pitch of the wrap is greater than the width of the tape in at least one portion of the device. In this way the aneurysm filling component comprises a coil structure with a polymeric covering that covers less than the full circumference of the coil in at least one section of the coil.

In one embodiment the polymeric component is a porous structure made by an electrospinning process. In one embodiment this porous structure comprises overlapping strands of a polymeric material. These strands may be monofilament or multifilament and may be laid down in a multiple layers.

The delivery device may comprise a catheter. The delivery device may further comprise a handle. The delivery device may further comprise a system for detachment of the aneurysm filling components so that they can be left permanently within the aneurysm. This detachment system may comprise an electrolytic detachment process or a melting process or a cutting process or a release mechanism. In an alternative embodiment the aneurysm filling components do not require detachment but are instead advanced as discrete elements through the catheter by means of a pusher. Said pusher may comprise a core wire or similar element, or may comprise a fluid such a saline injection through the catheter.

According to the disclosure there is also provided a method for the treatment of aneurysms, the method comprising:—

advancing a first aneurysm filling component through a delivery catheter to a target aneurysm;
deploying a first aneurysm filling component into the aneurysm so that it contacts at least a portion of the wall of the aneurysm;
advancing a second aneurysm filling component through a delivery catheter to the target aneurysm;
deploying the second aneurysm filling component into the aneurysm so that it at least partially sits within the space defined by the first aneurysm component.

Further embodiment of the above method may include methods wherein:

the first aneurysm filling component comprises a polymeric component having a porosity and/or cohesive energy density as previously described;
the second aneurysm filling component also comprises said polymeric component; the second aneurysm filling component comprises a bare coil without a polymeric component; the second aneurysm filling component comprises a coil structure with a polymeric component that is not ePTFE;
multiple first aneurysm filling components are deployed prior to deployment of one or more second aneurysm filling components.

In one aspect the disclosure provides a occlusion device comprising an embolic element and a pre-formed component which extends around at least a portion of the embolic element, the pre-formed component comprising a polymeric material having a cohesive energy density of less than 60 cal/cm³ and an internodal distance of less than 200 microns.

The pre-formed component may comprise a tape. The pre-formed component may comprise a micro-porous structure. The micro-porous structure may comprise a plurality of filaments.

In one embodiment the embolic element comprises a filament or wire.

The embolic element may comprise a coil. The coil may be formed into a tertiary shape.

In one case the embolic element comprises a coil and the pre-formed component comprises a tape which is wrapped around at least a portion of the coil.

In one embodiment the embolic element comprises at least a first wire and a second wire, the pre-formed component extending around at least a portion of the first wire and the second wire not having a pre-formed component.

At least one of the wires may comprise a shape memory material such as Nitinol. At least one of the wires may comprise a radiopaque material.

In one case the pre-formed component comprises tube. The tube may comprise a plurality of holes and/or slots through which the embolic element extends.

In one embodiment the embolic element comprises a wire and the pre-formed element comprises at least one strand, the wire and the strand being braided together.

The cohesive energy density of the polymeric component may be less than 50 cal/cm³, optionally less than 40 cal/cm³.

The internodal distance of the polymeric component may be less than 100 microns, optionally between 10 and 60 microns.

In one embodiment the polymeric component may comprise ePTFE.

### BRIEF DESCRIPTION OF THE DRAWINGS

The disclosure will be more clearly understood from the following description of an embodiment thereof, given by way of example only, with reference to the accompanying drawings, in which:

FIG. 1 is a schematic of an aneurysm treatment system of the disclosure;

FIGS. 2a and 2b are section views through an aneurysm being treated with a system of the disclosure;

FIG. 3 is an isometric view of an aneurysm filling component of the disclosure;

FIG. 4 is a side view of an aneurysm filling component of the disclosure;

FIG. 5 shows a portion of an aneurysm filling component of the disclosure;

FIG. 6 shows a portion of an aneurysm filling component of the disclosure;

FIG. 7 shows a portion of an aneurysm filling component of the disclosure;

FIG. 8 shows a portion of an aneurysm filling component of the disclosure;

FIG. 9 shows a portion of an aneurysm filling component of the disclosure;

FIG. 10 shows a section view of a portion of an aneurysm filling component of the disclosure;

FIG. 11 shows a section view of a portion of an aneurysm filling component of the disclosure;

FIG. 12 shows a section view of a portion of an aneurysm filling component of the disclosure;

FIG. 13 shows a partially completed aneurysm filling component of the disclosure;

FIG. 14 shows a partially completed aneurysm filling component of the disclosure;

FIG. 15 shows a section view of an aneurysm filling component of the disclosure;

FIG. 16 shows a section view of a portion of an aneurysm filling component of the disclosure;

FIG. 17 shows a section view of a portion of an aneurysm filling component of the disclosure;

FIG. 18 shows a section view of a portion of an aneurysm filling component of the disclosure;

FIG. 19 shows a section view of a portion of an aneurysm filling component of the disclosure;

FIG. 20 shows a section view of a portion of an aneurysm filling component of the disclosure;

FIG. 21 shows a portion of an aneurysm filling component of the disclosure;

FIG. 22 shows segments of various aneurysm filling components of the disclosure;

FIG. 23 shows a portion of an aneurysm filling component of the disclosure;

FIG. 24 shows a schematic of a material used in the disclosure; and

FIG. 25 shows an aneurysm filling component of the disclosure.

### DETAILED DESCRIPTION

An embolic occlusion device leveraging the cell in-growth properties of ePTFE is described for the treatment of aneurysms, arteriovenous malformations and other conditions where occlusion of a lumen or body cavity would be therapeutic. Expanded polytetrafluoroethylene (ePTFE) is a biocompatible material which exhibits cell ingrowth with connective tissue deposition in the interstices. Thus the material becomes anchored to the surrounding tissue. The cellular reaction is stereotypical and exhibits only low-grade inflammation. The primary reaction is one of mesenchymal cell (e.g. fibroblasts, fibrocytes, or smooth muscle cells) infiltration with collagen deposition. The well-organized cell and connective tissue deposition creates a firm scar tissue like formation. For treating aneurysms the ingrowth of cells with connective tissue deposition promotes adherence of the aneurysm wall to the embolic device thereby preventing continued growth or re-growth of the aneurysm as well as blocking blood flow into an aneurysm. In addition, biologically active substances can be loaded into the interstices of the ePTFE to enhance the fibrocellular reaction.

The disclosure described herein utilizes ePTFE in a form that results in embolic occlusion of a lumen or cavity. For example, in one embodiment coil wires could be coated with ePTFE that are delivered to the target site via a catheter system.

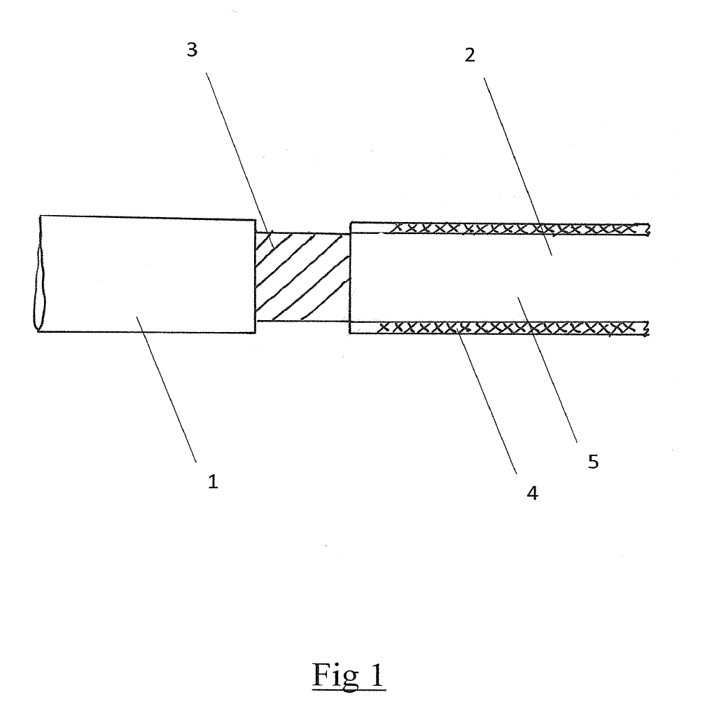

Referring to FIG. 1 of the drawings there is illustrated a system for the treatment of aneurysms comprising a delivery catheter 1 and an aneurysm filling component 2. The aneurysm filling component 2 further comprises a structural component 5 and a polymeric component 4, and is connected to a pusher element (not shown) within the deliver catheter through a detachment zone 3. The structural component and polymeric component may comprise any of the variants detailed in the proceeding figures and in previous descriptions.

A particularly suitable material for the polymeric component is expanded polytetrafluoroethylene or ePTFE. The cohesive energy density of PTFE is among the lowest of all polymers (38 cal/cm³—as described in “Polymer Science: A Materials Science Handbook” by A. D. Jenkins). For example Polyethylene has a cohesive energy density of approximately 60 cal/cm³ and Nylon has a cohesive energy density of approximately 200 cal/cm³. Because of this PTFE material has an extremely low coefficient of friction relative to most materials. ePTFE shares the low coefficient of PTFE and has the added benefit for this application of greatly increased porosity. This low coefficient of friction is highly advantageous in an aneurysm filling device as it enables the filling material to be advanced at a low force through a delivery catheter through the sometimes tortuous vasculature proximal to the site of the aneurysm to be treated. It also provides a highly atraumatic surface to the filling material, which enables cell migration and tissue in growth into the porous ePTFE structure to occur without an excessive inflammatory response. Thus it is highly advantageous to provide a porous material on the surface of the aneurysm filling element adjacent the vessel wall that has both an optimal porosity and a very low cohesive energy density.

The optimal porosity for an ePTFE material is an internodal distance of greater than 10 microns and less than 200 microns, preferably less than 100 microns, and most preferably between 10 and 60 microns. The preferable cohesive energy density of the material is less than 60 cal/cm³ and more preferably less than 50 cal/cm³ and most preferably less than 40 cal/cm³.

In order to achieve sufficient cell migration and tissue in growth for full integration of the polymer into the aneurysm wall it is necessary to have adequate depth (or wall thickness) of material. Preferably the material thickness will be at least three times the material porosity or internodal distance. Thus for an internodal distance of 200 microns a material thickness of at least 600 microns would be desirable, and for an internodal distance of 10 microns a material thickness of at least 30 microns would be desirable. Device wrapped profile is a key parameter for intracranial aneurysm coils, as these must be delivered through very low profile microcatheters, almost always with inner diameters of less than 900 microns, and typically with inner diameters of less than 600 microns. As ePTFE is a very soft and compliant material it requires a structural element to impart a degree of pushability to help in advancing it through a delivery catheter to the target site. Such a structural component may be a metallic coil and will itself occupy a significant percentage of the delivery catheter inner diameter. Thus it is desirable that the ePTFE material thickness is significantly less than the delivery catheter inner diameter, but at least three times greater than its internodal distance, and a material thickness of between 30 and 180 microns is preferred.

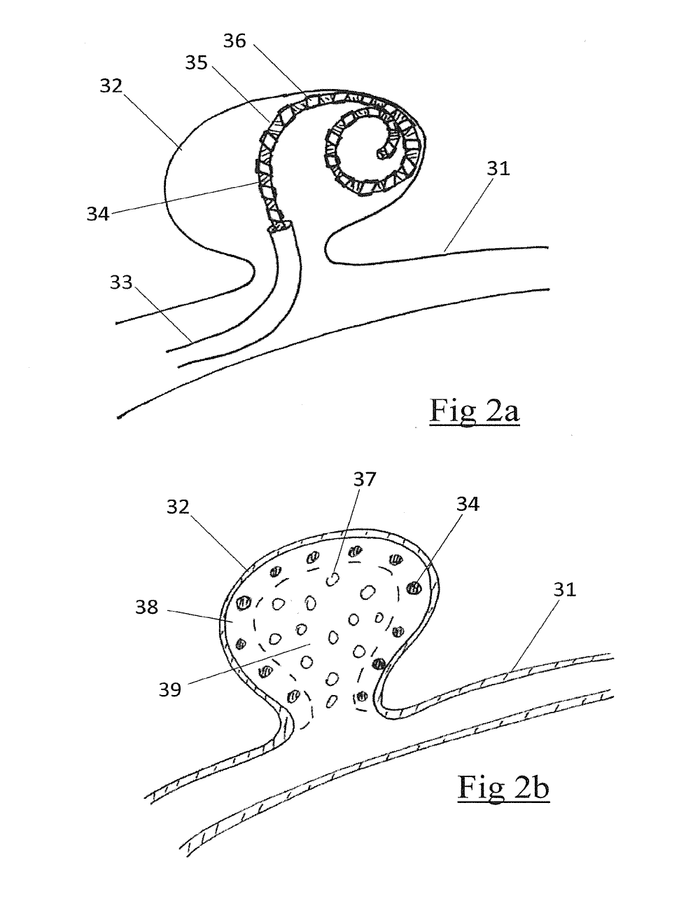

FIGS. 2a and 2b of the drawings illustrate an aneurysm 32 in a vessel segment 31 being treated with a system of this disclosure. In FIG. 2a aneurysm filling element 34 is shown being delivered through delivery catheter 33 into the internal lumen or cavity of the aneurysm 32. The aneurysm filling element 34 shown comprises a coil component 35 and a polymeric component 36 partially covering the coil, but the illustration is intended to describe a method of use of any of the aneurysm filling elements disclosed herein. FIG. 2b shows the same aneurysm after it has been filled with a plurality of aneurysm filling elements. Filling element 34 comprises a polymeric component and is positioned within a first space 38 adjacent the vessel wall in order to facilitate cell migration into the porous structure of the polymeric component and thus enable the aneurysm filling element to become incorporated into the aneurysm wall, strengthening the wall and minimizing the risk of subsequent rupture. Filling element 37 is positioned within a second space 39 within the first space 38, and may comprise a standard aneurysm filling coil, or may also be a polymeric covered coil of this disclosure.

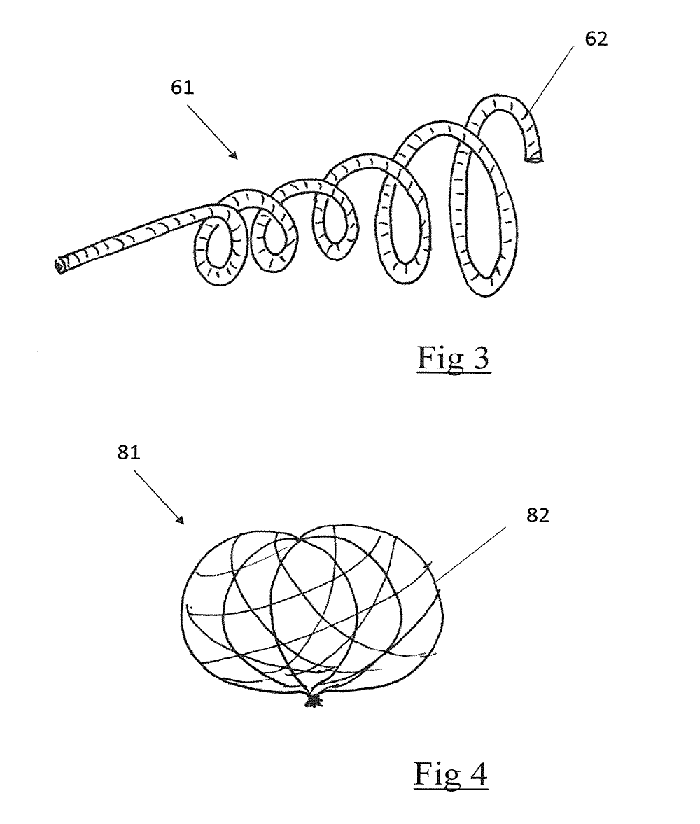

FIGS. 3 and 4 illustrate two examples of the many tertiary shapes into which the aneurysm filling components of this disclosure may be formed. The term tertiary shape is used simply because traditional aneurysm coils are often formed from a wire element (primary shape) which is formed into a long straight coil (secondary shape) and then this coil is formed into a three dimensional structure (tertiary shape) to assist in filling out the internal space within an aneurysm. Many different tertiary shapes are possible and different shapes may be advantageous for different purposes. For example a somewhat spherical shape may be advantageous for use as a framing coil which is intended to sit against or close to the aneurysm wall and helps to cover the neck of the aneurysm in order to help contain a filling coil which might be inserted after the framing coil or coils.

FIG. 3 illustrates a somewhat conical tertiary shape 61 into which the aneurysm filling component 62 has been formed.

FIG. 4 illustrates a somewhat spherical tertiary shape 81 into which the aneurysm filling component 82 has been formed. A tertiary shape such as this may be formed from a framework of fine Nitinol wires with a covering of polymeric material, wherein the polymeric material may be ePTFE and the covering may be applied in any of the ways disclosed elsewhere in this document.

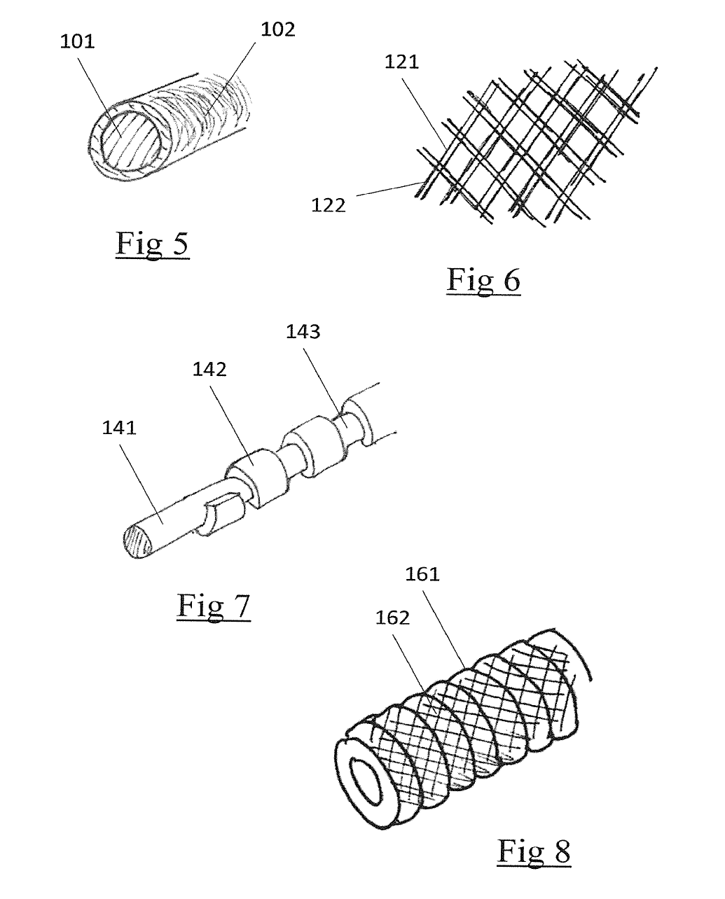

FIG. 5 illustrates a wire or filament 101 with a polymeric outer component 102. This polymeric outer may be a coating, which may be sprayed or dipped or painted or electrospun or otherwise applied. Alternatively this polymeric outer may be a membrane or tape which is wrapped around the outer surface of the wire or filament. In either case polymeric outer may be continuous over the surface of the wire or filament or may have discontinuities such that the entire outer surface of the wire or filament is not covered by the polymer.

FIG. 6 illustrates a schematic of a micro-porous structure comprising a plurality of filaments 121 spaced apart from each other and laid down in a first direction and a plurality of filaments 122 spaced apart from each other and laid down in a second direction, where the second direction is not parallel to the first direction. This type of porous structure could be made from a variety of polymer materials, but would preferably be made from a material with a low cohesive energy density as previously described. An electrospinning process could be employed to create this structure. This type of porous structure could be applied to a wire or filament as illustrated in FIG. 5 or to a coiled structure as illustrated in FIG. 8.

FIG. 7 illustrates a wire or filament 141 with a polymeric outer component 142 comprising a membrane or tape which is wrapped around the outer surface of the wire or filament. The tape or membrane may be wrapped such that one edge of the wind overlaps or contacts an adjacent edge, or preferably it may be wrapped with a gap 143 between edges created by employing a pitch wind that is greater than the tape width. Such a component can then be formed into a coil and set with a tertiary shape to create an aneurysm filling component of this disclosure.

FIG. 8 illustrates a coil component 161 with a micro-porous structure 162 comprising a plurality of filaments as illustrated in FIG. 6.

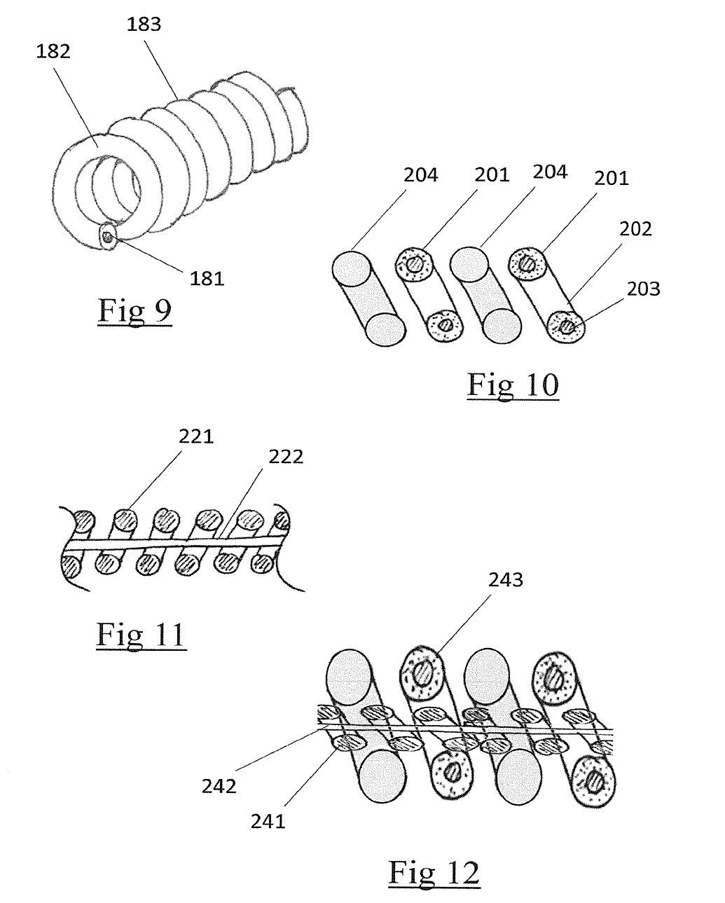

FIG. 9 illustrates a portion of an aneurysm filling component 183 of this disclosure comprising a core wire 181 with an outer polymer layer 182. This outer polymer layer 182 may be as described in relation to FIG. 5 or FIG. 7 or elsewhere in this document.

FIG. 10 illustrates a sectional view through a portion of an aneurysm filling component of this disclosure comprising a two start coil structure of a first wire component 204 and a second wire component 204. The first wire component 210 has a core wire 203 and an outer polymer layer 202. This outer polymer layer 202 may be as described in relation to FIG. 5 or FIG. 7 or elsewhere in this document. The second wire component is an uncoated wire structure.

In another embodiment the second wire component is identical to the first wire component.

In yet another embodiment the second wire component also has a polymeric component, but of a different construction to that of the first.

In yet another embodiment the coil is formed from three or more coil wires, at least one of which has a polymeric component as previously described.

In yet another embodiment the coil is formed from multiple coil wires, at least one of which is of a radiopaque material and at least one of which comprises Nitinol.

In yet another embodiment the aneurysm filling component comprises two layers of coils—an inner coil of a first material and an outer coil of a second material, wherein the first and second material may be the same or different and may be of a radiopaque material and/or of Nitinol. A polymeric component may be applied to either coil or to the outside of the two layer structure, or could be integrated into this structure.

FIG. 11 illustrates a sectional view through a portion of an aneurysm filling component of this disclosure comprising a coil structure 221 and an inner core wire 222. A polymeric component may be applied to either the coil wire or to the outside of the structure, or could be integrated into this structure.

FIG. 12 illustrates a sectional view through another structural component of an aneurysm filling component of this disclosure, in which there is an inner coil layer 241 with a an internal core wire 242 similar to the construct illustrated in FIG. 11, and an outer coil layer 243 similar to the construct illustrated in FIG. 10. A polymeric component may be applied to any of the coil wires or to the outside of the structure, or could be integrated into this structure.

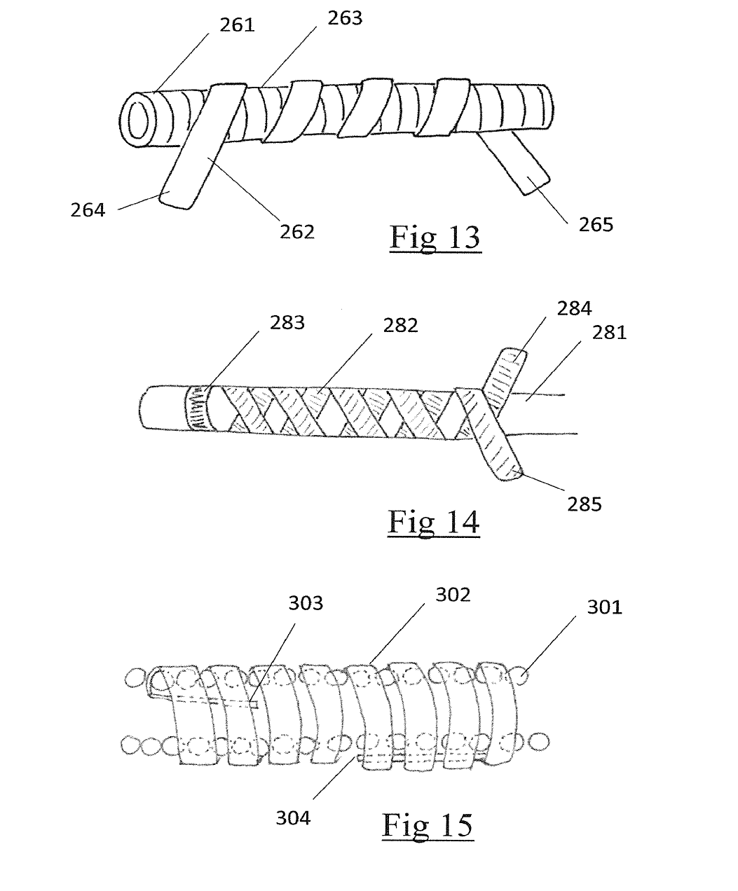

Referring to FIG. 13 of the drawings there is illustrated a partially constructed aneurysm filling component comprising a metallic coil element 261 around which has been wrapped a polymeric tape element 262, such that the pitch of the tape wind is greater than the width of the tape leaving uncovered areas 263 between the tape winds. Tape ends 264 and 265 may be integrated into the component in a number of different ways such as adhesive bonding, heat staking, tying off or wrapping under itself or inside the coil structure (as illustrated in FIG. 15).

Referring to FIG. 14 of the drawings there is illustrated a partially constructed aneurysm filling component comprising a metallic coil element 281 around which has been wrapped a polymeric tape element 282, which is wrapped in an overlapping or braided fashion such that end 283 is wrapped tight around the coil and ends 284 and 285 are positioned adjacent to each other. Thus ends 284 and 285 can be joined to each other by tying or bonding or heat staking or other means, or can be wrapped inside the coil structure as illustrated in FIG. 15.

Referring to FIG. 15 of the drawings there is illustrated a partially constructed aneurysm filling component comprising a metallic coil element 301 around which has been wrapped a polymeric tape element 302, of which ends 303 and 304 have been finished off by wrapping inside the coil structure and under the tape wind respectively.

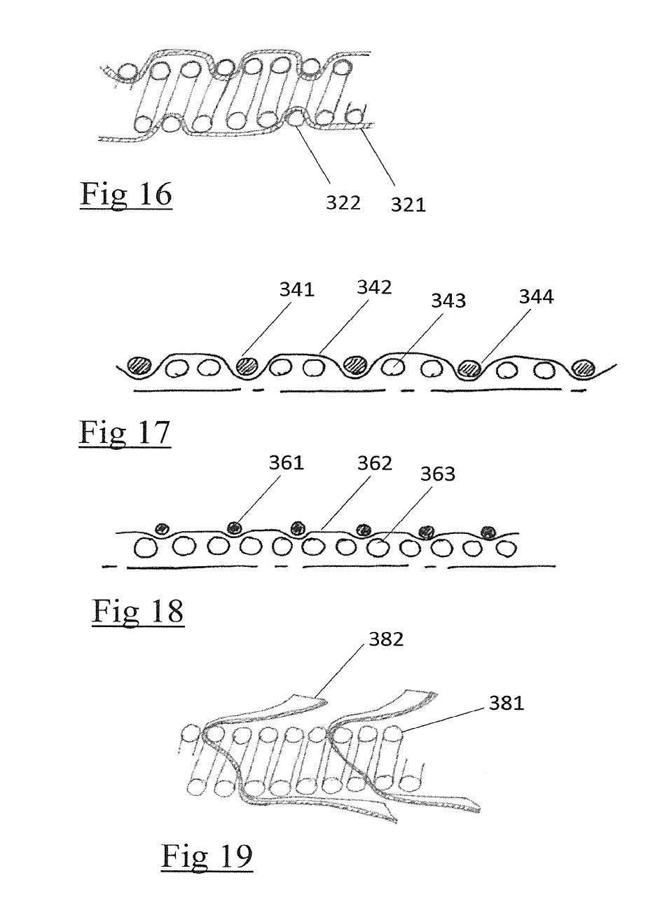

Referring to FIG. 16 of the drawings there is illustrated a section view through an aneurysm filling component comprising a metallic coil element 322 through which has been wrapped a polymeric tape element 321, such that polymeric tape element sits inside the coil structure at intervals to hold it in place.

Referring to FIG. 17 of the drawings there is illustrated a partial section view through an aneurysm filling component of a similar construct to that described in FIG. 16, but in this case the assembly of the device has been significantly simplified by employing two separate coils. Polymeric tape 342 has been applied to the outer surface of a first coil 343, after which a second coil 341 has been coiled around the resultant structure. The second coil 341 sits in gaps 344 left between winds of the first coil 343, and compresses the tape to sit within these gaps, thus trapping the tape securely inside the overall coil structure.

Referring to FIG. 18 of the drawings there is illustrated a partial section view through an aneurysm filling component of a similar construct to that described in FIG. 17, but in this case the assembly of the device has been even further simplified. Polymeric tape 362 has been applied to the outer surface of a first coil 363, after which a second coil 361 has been coiled around the resultant structure, thus trapping the tape securely inside the overall coil structure.

Referring to FIG. 19 of the drawings there is illustrated a section view through an aneurysm filling component comprising a metallic coil element 381 through which has been wrapped segments of polymeric tape element 382, such that polymeric tape segments are threaded through the coil structure at intervals to hold it them in place. The resultant “sails” of polymer will wrap down adjacent the coil for delivery through the microcatheter, but can occupy a greater volume when deployed within the aneurysm, providing a large surface area for cell migration and tissue ingrowth.

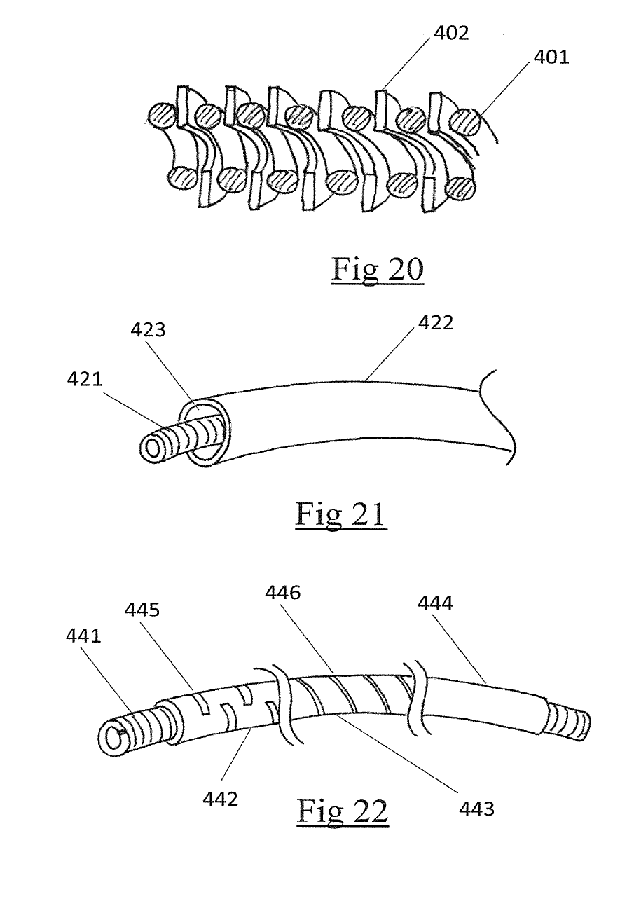

Referring to FIG. 20 of the drawings there is illustrated a section view through an aneurysm filling component comprising a metallic coil element 401 through which has been wrapped a polymeric tape element 402, such that polymeric tape element is wrapped between the coils of element 401. Thus the majority of the tape surface is aligned generally normal to the central axis of the coil, rather than generally parallel to the axis.

Referring to FIG. 21 of the drawings there is illustrated an aneurysm filling component comprising a polymeric tube component 422 within which is positioned a metallic coil element 421. The polymeric tube material may be an ePTFE material as previously discussed, or have the porosity and/or cohesive energy density properties previously discussed. In one embodiment (as shown) there is a space 423 between the polymeric tube inner surface and the coil outer surface, so that the coil and tube surfaces are free to move relative to one another. This spacing means that the polymeric tube can crease or kink to adopt a tight bend radius without significantly adding to the lateral or bend stiffness of the coil structure. This enables the structure to adopt an efficient packed configuration within an aneurysm.

Having the ePTFE material applied to the coil in the form of a tube is very advantageous in that it eliminates the finishing challenges associated with applying a tape to the coil, but it presents a significant material processing challenge. This is because ePTFE is typically manufactured in sheet form, and calendaring and stretching processes are employed to create the desired porous structure. While it is possible to do this to create a tube rather than a sheet it is very difficult to create a sufficient degree of porosity in a tubular form. One solution to this challenge is to create the tubular ePTFE structure from sheet material by first wrapping it into a tube and then applying a secondary process to join or seal the resultant seam.

Referring to FIG. 22 of the drawings there is illustrated various segments of an aneurysm filling component comprising a metallic coil element 441 over which sits a polymeric tube element. The polymeric tube element 442 has slots 445 removed from it to provide the structure with freedom to bend and pack efficiently within an aneurysm. The polymeric tube element 443 has a continuous spiral slot 446 removed from it to provide the structure with freedom to bend and pack efficiently within an aneurysm. The polymeric tube element 444 has no removed material.

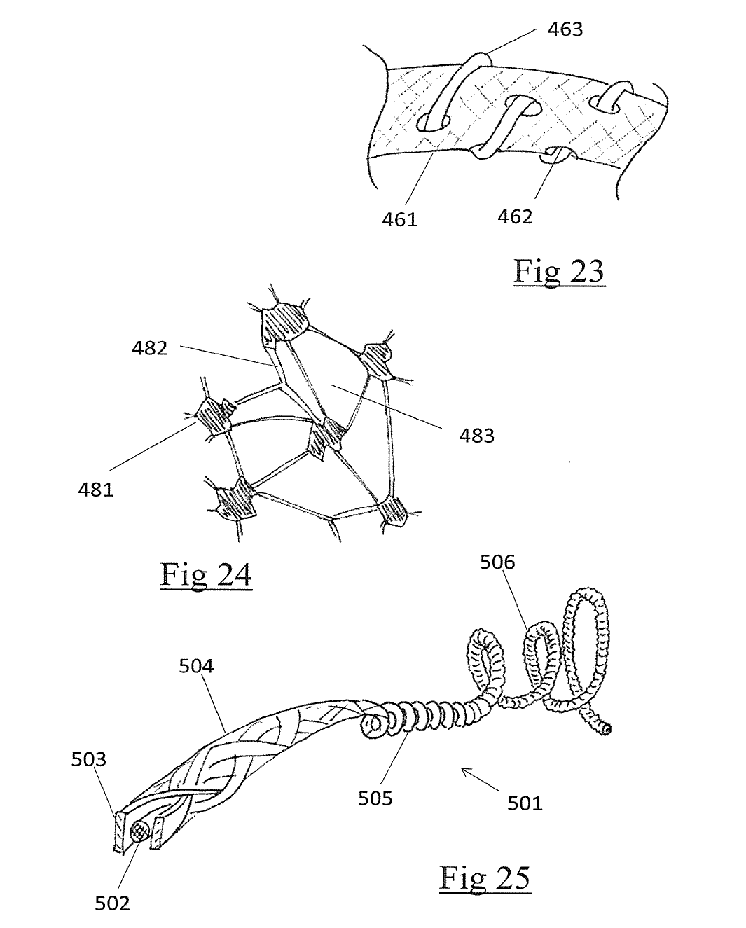

Referring to FIG. 23 of the drawings there is illustrated an aneurysm filling component comprising a polymeric tube component 461 comprising a plurality of openings 462 through which is threaded one or more metallic wire elements 463. The polymeric tube may comprise an ePTFE tube and the holes may be laser cut or otherwise pressed, cut or machined out. The wire element may comprise a radiopaque material such as Platinum or an alloy of this or a similar material.

FIG. 24 shows a schematic representation of the porous nature of ePTFE material. Nodes 481 of solid PTFE are connected to each other by fibrils 482, creating a three dimensional porous structure with large open areas 483 between the nodes. It is into these open areas that cells can migrate with resultant tissue ingrowth.

Referring to FIG. 25 of the drawings there is illustrated another aneurysm filling component 501 of this disclosure comprising a wire element 502 and one or more polymeric strand elements 503. The wire and polymeric elements are intertwined or braided to form a primary cable element 504, which is in turn coiled to form a secondary coil shape 505, which is itself formed into a tertiary three dimensional form 506. The polymeric material may compromise ePTFE and/or may comprise the preferred attributes of cohesive energy density and internodal distance previously discussed. The wire element may compromise a radiopaque material such as Platinum or an alloy of this or other such high atomic number material.

The present disclosure exhibits at least three advantages over the coating approaches of earlier disclosures. One is that the ePTFE (e.g. as a coating for an embolic coil) is permanent. It can't slough during delivery and does not dissolve as a function of time. Bioresorbable coatings typically dissolve (via a hydrolytic process) before the aneurysm has been healed by the deposition of granulation tissue. The inadequate healing exposes the aneurysm to risk of regrowth or rupture and has been implicated in coil compaction. An ePTFE coated device would not suffer these limitations because it is a permanent coating. In addition, ePTFE is inherently “slippery” thereby precluding it from impeding performance within the microcatheter during delivery. Furthermore, because cells invariably migrate into the interstices or pores of the ePTFE, the coated coils (or similar embodiment) will anchor to the aneurysm wall thereby inhibiting or preventing aneurysm regrowth.

It will be apparent from the foregoing description that, while particular embodiments of the present disclosure have been illustrated and described, various modifications can be made without departing from the spirit and scope of the disclosure. For example, while embodiments may refer to particular features, the disclosure includes embodiments having different combinations of features. The disclosure also includes embodiments that do not include all of the specific features described.

The disclosure is not limited to the embodiments hereinbefore described which may be varied in construction and detail.

1. A system for the treatment of an aneurysm, the system comprising:—
an aneurysm filling component and a delivery device;
the aneurysm filling component comprising a polymeric component;
the polymeric component having a cohesive energy density of less than 60 cal/cm³ and an internodal distance of less than 200 microns;
the delivery device comprising a catheter;
and the aneurysm filling component configured to be delivered through the catheter to a target aneurysm.

2. The system as claimed in claim 1 wherein the aneurysm filling component comprises one or more structural components.

3. The system as claimed in claim 2 wherein at least one of the structural components comprises a coil.

4. The system as claimed in claim 1 wherein the cohesive energy density of the polymeric component is less than 50 cal/cm³.

5. The system as claimed in claim 1 wherein the cohesive energy density of the polymeric component is less than 40 cal/cm³.

6. The system as claimed in claim 1 wherein the internodal distance of the polymeric component is less than 100 microns.

7. The system as claimed in claim 1 wherein the internodal distance of the polymeric component is between 10 and 60 microns.

8. The system as claimed in claim 1 wherein the polymeric component comprises ePTFE.

9. The system as claimed in claim 1 wherein the polymeric component is in the form of a tape.

10. The system as claimed in claim 9 wherein the tape is at least partially wrapped around a coil structure.

11. The system as claimed in claim 10 wherein the pitch of the wrap is greater than the width of the tape in at least one portion of the device.

12. The system as claimed in claim 1 wherein the aneurysm filling component comprises a coil structure having a polymeric covering that covers less than the full circumference of the coil in at least one section of the coil.

13. A method for the treatment of an aneurysm, the method comprising:—
advancing a first aneurysm filling component through a delivery catheter to a target aneurysm;
deploying a first aneurysm filling component into the aneurysm so that it contacts at least a portion of the wall of the aneurysm;
advancing a second aneurysm filling component through a delivery catheter to the target aneurysm; and
deploying the second aneurysm filling component into the aneurysm so that it at least partially sits within the space defined by the first aneurysm component.

14. A method as claimed in claim 13 wherein the first aneurysm filling component comprises a polymeric component having a cohesive energy density of less than 60 cal/cm³ and an internodal distance of less than 200 microns.

15. An occlusion device comprising an embolic element and a pre-formed component which extends around at least a portion of the embolic element, the pre-formed component comprising a polymeric material having a cohesive energy density of less than 60 cal/cm³ and an internodal distance of less than 200 microns.

16. The occlusion device as claimed in claim 15 wherein the pre-formed component comprises a tape.

17. The occlusion device as claimed in claim 15 wherein the pre-formed component comprises a micro-porous structure.

18. The occlusion device as claimed in claim 17 wherein the micro-porous structure comprises a plurality of filaments.

19. The occlusion device as claimed in claim 15 wherein the embolic element comprises a filament or wire.

20. The occlusion device as claimed in claim 15 wherein the embolic element comprises a coil.

21. The occlusion device as claimed in claim 20 wherein the coil is formed into a tertiary shape.

22. The occlusion device as claimed in claim 15 wherein the embolic element comprises a coil and the pre-formed component comprises a tape which is wrapped around at least a portion of the coil.

23. The occlusion device as claimed in claim 15 wherein the embolic element comprises at least a first wire and a second wire, the pre-formed component extending around at least a portion of the first wire and the second wire not having a pre-formed component.

24. The occlusion device as claimed in claim 23 wherein at least one of the wires comprises a shape memory material such as Nitinol.

25. The occlusion device as claimed in claim 23 wherein at least one of the wires comprises a radiopaque material.

26. The occlusion device as claimed in claim 15 wherein the pre-formed component comprises a tube.

27. The occlusion device as claimed in claim 26 wherein the tube comprises a plurality of holes through which the embolic element extends.

28. The occlusion device as claimed in claim 15 wherein the embolic element comprises a wire and the pre-formed element comprises at least one strand, the wire and the strand being braided together.

29. The occlusion device as claimed in claim 15 wherein the cohesive energy density of the polymeric component is less than 50 cal/cm³.

30. The occlusion device as claimed in claim 15 wherein the cohesive energy density of the polymeric component is less than 40 cal/cm³.

31. The occlusion device as claimed in claim 15 wherein the internodal distance of the polymeric component is less than 100 microns.

32. The occlusion device as claimed in claim 15 wherein the internodal distance of the polymeric component is between 10 and 60 microns.

33. The occlusion device as claimed in claim 15 wherein the polymeric component comprises ePTFE.
